SEQUENCE LISTING (1 sequence)

SEQ ID NO: 1 - DNA, 1524 bp, Christensenella minuta
  source 1..1524: mol_type = other DNA
agagtttgatcctggctcaggacgaacgctggcggcgtgcttaacacatgcaagtcgaacgaggttgccctttgtgaatccttcgggaggaactgtgggtataccgagtggcggacgggtgagtaacgcgtgagcaacctgccctgcaacgggggacaacagttggaaacgactgctaataccgcatgagaccacgaaaccgcatggttttgaggtaaaaggatttattcgatgcaggatgggctcgcgtcccattagatagttggtgaggtaacggcccaccaagtcaacgatgggtagccgacctgagagggtgatcggccacactggaactgagacacggtccagactcctacgggaggcagcagtggggaatattgggcaatgggggaaaccctgacccagcaacgccgcgtgagggaagaaggtcttcggattgtaaacctttgtcctatgggacgaaagaaatgacggtaccataggaggaagctccggctaactacgtgccagcagccgcggtaatacgtagggagcaagcgttgtccggaattactgggcgtaaagggtgcgtaggtggtcatgtaagtcagatgtgaaagaccggggcttaaccccggggttgcatttgaaactgtgtgacttgagtacaggagagggaagtggaattcctagtgtagcggtgaaatgcgtagatattaggaggaacaccagtggcgaaggcgactttctggactgtaactgacactgaggcacgaaagcgtggggagcaaacaggattagataccctggtagtccacgccgtaaacgatggatactaggtgtggggcccgatagggttccgtgccgaagcaaacgcattaagtatcccgcctggggagtacgatcgcaaggttgaaactcaaaggaattgacgggggcccgcacaagcagcggagcatgtggtttaattcgaagcaacgcgaagaaccttaccaaggcttgacatcctctgacgactgtagagatacagtttcccttcggggcagagagacaggtggtgcatggttgtcgtcagctcgtgtcgtgagatgttgggttaagtcccgcaacgagcgcaacccttattgctagttgccagcgcgtaaaggcgggaactctagtgagactgccggggacaactcggaggaaggtggggacgacgtcaaatcatcatgccccttatgtcttgggctacacacgtgctacaatggccggtacaaagggcagcgaacccgtaaggggaagcgaatctcaaaaagccggtcccagttcggattgtgggctgcaacccgcccacatgaagtcggagttgctagtaatcgcgaatcagcatgtcgcggtgaatgcgttcccgggccttgtacacaccgcccgtcacaccacggaagttgggagcacccgaagccagtggcttaaccgtaaggagagagctgtcgaaggtgagatcaatgactggggtgaagtcgtaacaaggtagccgtatcggaaggtgcggctggatcacct

(19) United States
(12) Patent Application Publication
(10) Pub. No.: US 2025/0127824 A1
(43) Pub. Date: Apr. 24, 2025

(54) BACTERIAL STRAIN USED IN THE PREVENTION AND/OR TREATMENT OF INFLAMMATORY PATHOLOGIES

(71) Applicant: VERB BIOTICS, BOSTON, MA (US)

(72) Inventors: Georges RAWADI, PARIS (FR); Sandrine Paule CLAUS, BORDEAUX (FR); Héloïse TUDELA, CENON (FR); Katy Nicole LECORF, MERIGNAC (FR); Karima RELIZANI, BORDEAUX (FR); Frédéric ELUSTONDO, MERIGNAC (FR)

(21) Appl. No.: 18/691,975

(22) PCT Filed: Sep. 16, 2022
(86) PCT No.: PCT/EP2022/075781

§ 371 Date: Apr. 2, 2024

(30) Foreign Application Priority Data
Sep. 16, 2021 (FR) FR2109727

(51) Int. Cl. A61K 35/741; A61K 45/06; A61P 1/00

(52) U.S. Cl. CPC A61K 35/741; A61K 45/06; A61P 1/00

(57) ABSTRACT

The present invention relates to a novel bacterial strain belonging to the genus Christensenella, a composition comprising the same and uses thereof, in particular as a medicament in the treatment and/or prevention of diseases such as metabolic diseases and/or inflammatory diseases.

### TECHNICAL FIELD

The invention relates to a novel bacterial strain belonging to the genus Christensenella, a composition comprising the same and uses thereof, in particular as a medicament in the treatment and/or prevention of diseases such as metabolic diseases and/or inflammatory diseases.

### PRIOR ART

Our intestinal microbiota or “intestinal flora” consists of a set of non-pathogenic bacteria, viruses, parasites and fungi, or 10¹² to 10¹⁴ microorganisms present in the digestive tract of each individual, which is 2 to 10 times more than the number of cells in our body. Therefore, its role has been increasingly studied in recent years. In 2012, a microbiologist identified and cultivated from human feces a new species: Christensenella minuta, belonging to gram-negative Clostridia. (Morotomi et al., Description of Christensenella minuta gen. nov, sp. nov, isolated from human faeces, which forms a distinct branch in the order Clostridiales, and proposal of Christensenellaceae fain. nov, International Journal of Systematic and Evolutionary Microbiology (2012), 62, 144-149).

It is now accepted by the scientific community that the human intestinal microbiota is linked to the appearance of certain “non-transmissible” pathologies associated with dysbiosis of the microbiota, such as obesity, diabetes, metabolic diseases, intestinal inflammatory diseases, or even depression. However, the prevalence of these diseases does not cease to increase, without any satisfactory treatment.

Chronic inflammatory bowel diseases encompass diseases linked to inflammation of the intestine that are chronic in nature. Crohn's disease and ulcerative colitis are the main ones. These are recent, multi-factor diseases whose origin is unknown. In a certain number of cases, the appearance of these diseases could be linked to genetic predisposition, to the environment, or to microbiota. These diseases all have the particularity of exhibiting inflammation of the intestine, which induces a modification of the microbiota and then maintains inflammation.

In the context of Crohn's disease, inflammation is most often grouped in the ileum and colon. Ulcerative colitis (UC) more particularly affects the distal end of the digestive tract, that is to say the colon and the rectum. Its incidence remained stable, at 4.4 case per 100,000 inhabitants in France, whereas that of Crohn's disease increased by 5.3 to 7.6 cases per 100,000 inhabitants in France between 1988 and 2014.

These are disabilities for which no curative treatment is available and whose prevalence has increased greatly in Western countries. These diseases act in spurts, the remission period of which varies in length. The objective of current treatments is to prolong the duration of the remissions as possible and therefore to improve the quality of life of patients despite life treatments.

Despite progress in understanding, and the diagnosis of these pathologies, modern medicine does not have a satisfactory curative solution, which causes major public health problems, in particular in adolescents, in whom prevalence has greatly increased in recent years.

There is therefore a strong medical need for a product capable of acting on the intestinal microbiota, in particular in the prevention and treatment of chronic inflammatory bowel diseases.

One objective of the present invention is therefore to provide a solution that is simple, effective and economical to restore a healthy intestinal microbiota and thus treat the dysbiosis of intestinal microbiota, in particular to treat chronic inflammatory diseases, preferentially while avoiding the administration of a lifetime treatment and thus roll back the prevalence of these diseases.

### SUMMARY OF THE INVENTION

To meet this objective, the inventors have identified, from among the multitude of bacteria present in the intestinal microbiota and from already-known strains belonging to the species Christensenella minuta, a new specific bacterial strain of Christensenella minuta filed with the Institut Leibniz DSMZ (Deutsche Sammlung von Mikroorganismen und Zelikulturen GmbH, meaning German Collection of Microorganisms and of Cell Cultures GmbH) under number DSM 33715, particularly suitable for the prevention and/or treatment of an intestinal dysbiosis, preferentially chronic inflammatory bowel diseases.

Thus, the invention relates to a bacterial strain of Christensenella minuta deposited under number DSM 33715, having the 16S rDNA sequence, the sequence SEQ ID NO:1, thus all strains comprising a nucleotide sequence having at least 99.90% ANI (Average Nucleotide Identity) with the nucleotide sequence of the genome of the bacterial strain DSM 33715.

The invention also relates to the supernatant of the culture of the bacterial strain according to the invention but also to a composition comprising (i) at least the bacterial strain according to the invention and/or (ii) the supernatant of the culture according to the invention and (iii) at least one acceptable excipient. Said acceptable excipient preferentially being a pharmaceutically acceptable excipient when it is a product intended to be used as a medicament for human use or veterinary use.

Finally, the invention is particularly suitable for using said bacterial strain, said supernatant, or said composition according to the invention as a medicament. Indeed, the present invention is particularly suitable for the prevention and/or treatment of pathologies called “non-transmissible” and associated with dysbiosis of intestinal microbiota or diseases associated with dysbiosis of intestinal microbiota, in particular diseases such as metabolic diseases, inflammatory diseases and others which will be detailed below.

Preferentially, the invention aims to prevent and/or treat chronic inflammatory bowel diseases, more preferentially Crohn's disease, or ulcerative colitis.

Finally, the invention also relates to a non-therapeutic use of the bacterial strain according to the invention, and/or of the supernatant according to the invention, and/or of the composition according to the invention for maintaining and/or reinforcing the intestinal microbiota and/or supporting the diversity of the intestinal microbiota and/or promoting the increase of beneficial bacteria in the intestinal microbiota in a healthy subject.

Other features and advantages will become apparent from the detailed description of the invention, the examples and the figures that follow.

### BRIEF DESCRIPTION OF THE FIGURES

FIG. 1A shows the image of a Christensenella minuta bacterium according to the invention produced by transmission electron microscopy in negative coloring (magnification: 36kX).

FIG. 1B shows a MALDI spectrum characteristic of a pure culture of a Christensenella minuta strain according to the invention.

FIG. 2A shows the immunomodulatory effect of the bacterium DSM 33715 on PBMCs cells (3 healthy donors tested)

FIG. 2B shows the anti-inflammatory effect of the bacterium DSM 33715 on a THP-1 line differentiated into macrophages of the MO type by treatment with PMA (Phorbol 12-myristate 13-acetate).

FIG. 3 shows the immuno-modulating effect of the bacterium DSM 33715 and of its supernatant on the IL-8 producing HT-29 colon adenocarcinoma line after stimulation with TNF-α.

FIG. 4A shows the anti-inflammatory effect of DSM 33715 on the NF-0B inflammation signaling pathway. Activation of the NF-0B pathway by DSM 33715 in HT-29 cells transfected with a reporter system and stimulated by TNF-α, measured by luciferase activity and normalized by Renilla luminescence.

FIG. 4B shows the anti-inflammatory effect of DSM 33715 on the JAK/STAT inflammation signaling pathway. Activation of the JAK/STAT pathway by DSM 33715 in HT-29 cells transfected with a reporter system and stimulated by IFN-γ, measured by luciferase activity and normalized by Renilla luminescence.

FIG. 5 shows the immuno-modulating effect of the supernatant of DSM 33715 on the IL-8 producing HT-29 colon adenocarcinoma line after stimulation with TNF-α, in the presence of an anti-inflammatory agent used in therapeutics, 5-ASA or Budesonide.

FIG. 6 shows the anti-inflammatory effect of DSM 33715 associated or not with Tofacitinib on the modulation of the JAK/STAT inflammation signaling pathway induced by IFN-γ. The JAK/STAT pathway is activated in HT-29 cells transfected with a reporter system and stimulated by IFN-γ, measured by luciferase activity and normalized by Renilla luminescence.

FIG. 7 shows the effect of the DSM 33715 strain on the integrity of the intestinal barrier by measuring the transepithelial electrical resistance. The condition PBS-Glycerol+/−TNF-α (control) corresponds to the resuspended bacterial medium.

FIG. 8A is a histogram representing the average length of the colons, at the end of the experiment. The data are represented by the +/−SEM mean in centimeters for the length and in grams for the weight of the colon.

FIG. 8 shows the inflammation of the colon at the macroscopic level calculated by the Wallace score.

FIG. 8C shows the inflammation at the histological level calculated by the Ameho score. The data shown are the +/−SEM means. MGG Coloration of colon sections for evaluation of the histological score (Ameho)

FIG. 8D shows the IL-1P (panel A) and Lipocalin-2 (panel B) levels in the colon. The data shown correspond to the +/−SEM mean in ng/mg of proteins.

FIG. 9 shows the intensity of the inflammation and the lesions of the colon that were evaluated at the macroscopic level according to the Wallace score (panel A) and the intensity of the inflammatory lesions at the tissue level evaluated according to the histological score (panel B).

### DETAILED DESCRIPTION OF THE INVENTION

### Definition

The term “bacterium” in the sense of the invention is understood to mean a single-cell microorganism capable of being reproduced by cell division. The bacteria are classified by family, genus, species. Each bacterial species comprises a diversity of bacterial strains. The bacterial strain according to the invention belongs to the family Christensenellaceae, the genus Christensenella and the species minuta. Therefore, “bacterial strain” or “strain” within the meaning of the invention is a specific bacterial strain but also all the bacteria derived from the strain or obtained from the strain or corresponding to the bacterial strain and having the same metabolic functions, for example at least one bacterium removed from a colony derived from the strain. For the purposes of the invention, “bacterium (bacteria) according to the invention” is therefore also a bacterial strain according to the invention.

“Derived bacterial strain” or “mutant strain” or “derived strain” within the meaning of the invention is understood to mean a bacterial strain having a strong similarity with the bacterial strain deposited under the number DSM 33715. Preferentially, the strain comprises a nucleotide sequence having at least 99.90% ANI identity with the nucleotide sequence of the bacterial strain DSM 33715, more preferentially at least 99.91%, at least 99.92%, at least 99.93%, at least 99.94%, at least 99.95%, at least 99.96%, at least 99.97%, at least 99.98%, or at least 99.99% ANI identity.

The term “supernatant” within the meaning of the invention is understood to mean the culture supernatant of the bacterial strain according to the invention optionally comprising cellular components of said strain and/or cell debris of said strain, and/or metabolites and/or molecules secreted by said strain.

“Prevention” within the meaning of the invention is understood to mean the reduction to a lesser degree of the risk or the probability of occurrence of a given phenomenon, that is, in the context of the present invention, dysbiosis of the intestinal microbiota and associated diseases, more preferentially chronic inflammatory diseases, for example Crohn's disease or ulcerative colitis.

“Treatment” within the meaning of the invention is understood to mean a decrease in the progression of the disease, a stabilization, an inversion or regression, or even an interruption or inhibition of the progression of dysbiosis of the intestinal microbiota and associated diseases, more preferentially chronic inflammatory diseases, for example Crohn's disease or ulcerative colitis. In the context of the invention, these terms also apply to one or more symptoms of said diseases of the present invention.

For the purposes of the invention, the term “physiologically acceptable medium” refers to a medium which is compatible with the body of the individual to which said composition is to be administered. It may involve, for example, a non-toxic solvent such as water, buffer, saline solutions. In particular, said medium is compatible with oral administration.

For the purposes of the invention, the term “acceptable excipient” is intended to mean any compound making it possible to facilitate the formulation of the composition and not modifying the nature of the biological activity of the active ingredient. An acceptable excipient may be a solvent, buffer, saline solution, plasticizer, lubricant, dispersion medium, absorption retarding agent, flow agent, isotonic agent. Preferentially, it may be pharmaceutically acceptable excipients that are selected according to the pharmaceutical form and the desired mode of administration, among the usual excipients known to a person skilled in the art and suitable for human and/or veterinary use. The excipient will thus be chosen as a function of the administration route, for example suitable for oral, intravenous, intramuscular, topical administration etc.

The term “disease associated with dysbiosis of the intestinal microbiota” within the meaning of the invention is understood to mean diseases associated with dysbiosis of the intestinal microbiota such as certain metabolic diseases, for example obesity-related diseases including obesity, diabetes, NASH, hepatic or pancreatic steatosis, inflammatory diseases, in particular chronic inflammatory bowel diseases, for example Crohn's disease, ulcerative colitis, diverticulitis, gastric diseases, pancreatic diseases, or irritable bowel syndrome; but also chronic diseases associated with dysbiosis, cardiac and vascular diseases, cancers, for example cancers linked to metabolism.

Within the meaning of the invention, “dysbiosis of the intestinal microbiota” is understood to mean an imbalance of this microbiota which could promote the initiation, maintenance, or severity of inflammation. As an example, in about 5% of patients suffering from Crohn's disease, there is for example a family of Escherichia coli (AIEC) that adheres more to the cells of the intestinal wall and is more invasive than the usual strains, which facilitate a local inflammatory reaction. Potential causes of dysbiosis could be of food origin (fat and sugar, without fiber, which limits the bacteria producing short beneficial chain fatty acids), infectious (episodes of infectious gastroenteritis), or environmental (repeated antibiotic treatments, insufficient exposure to pathogens during childhood).

The term “anti-inflammatory agent” is understood to mean, within the meaning of the invention, a molecule, an active ingredient, a medicament, or a pharmaceutical composition intended to combat inflammation. In particular, it is understood to mean a group of medicaments intended to treat an inflammatory reaction and diseases resulting therefrom such as chronic inflammatory bowel diseases. By way of example, mention may be made, as anti-inflammatory medicaments, of corticoids (or steroidal anti-inflammatories or anti-inflammatories) and non-steroidal anti-inflammatories.

For the purposes of the invention, the term “healthy subject” is intended to mean a subject who is not ill and does not suffer from a disease, in particular does not suffer from a disease chosen from chronic diseases and/or obesity and/or metabolic diseases and/or inflammatory diseases and/or cancers.

### Bacterial Strain or Supernatant According to the Invention

The inventors have discovered the bacterial strain of Christensenella minuta filed under number DSM 33715, then isolated from feces of a healthy human donor and demonstrated, in models in vitro, anti-inflammatory effects, in particular an immuno-modulating effect such as the induction of an IL-10 production, an upper anti-inflammatory cytokine, and a decrease in certain signaling pathways such as the NF-0B pathway or the JAK/STAT pathway. Results on a pre-clinical model of colitis induced by TNBS in rats make it possible to confirm the anti-inflammatory effects of the strain, in particular on chronic inflammatory bowel diseases.

The inventors have also demonstrated that said bacterial strain DSM 33715 makes it possible to restore or reinforce and therefore preserve the integrity of the intestinal barrier Finally, an action synergy was established when the strain is used in combination with anti-inflammatory agents used in clinical settings such as Pentasa (5-ASA), Tofacitinib or Budesonide, thus making it possible to envisage the administration of smaller doses of such anti-inflammatory agents known for their toxicity and their undesirable effects from patients.

The present invention therefore relates to a bacterial strain of Christensenella minuta deposited under number DSM 33715, referred to below as strain DSM 33715, with 16S rDNA, the sequence SEQ ID NO:1.

16S ribosomal RNA (16S rRNA) is ribosomal RNA constituting the small subunit of prokaryotic ribosomes. The genes coding for this RNA are called 16S rDNA. The 16S rDNA sequence is very widely used in phylogenesis due to its very conserved structure, which makes it possible to reconstruct the evolutionary history of organisms and in particular of prokaryotes and bacteria.

The percentage of identity of the 16S rDNA sequence between two bacterial strains, more particularly between two strains of Christensenella minuta, can be determined by the so-called BLAST method, which is a heuristic search method well known to a person skilled in the art. It makes it possible to find similar regions between two or more nucleotide or amino acid sequences, and to carry out an alignment of these homologous regions.

However, the 16S rDNA sequence does not always allow differentiation of two strains of the same species having different properties, for example anti-inflammatory properties. Therefore, a person skilled in the art by their general knowledge is able to characterize a bacterial strain according to other parameters such as the calculation of phylogenetic distance based on the complete genome (ANI), the size of the genome, the CDS number (Coding DNA Sequence), but also the identification of genes specific to said strain of interest.

For the purposes of the invention, “ANI” refers to the percentage of average identity of the nucleotides calculated from the two-by-two comparison of all the genome sequences between the two bacterial strains. According to the general knowledge well known to a person skilled in the art, the genomic DNA can be extracted from said strain of interest deposited with the DSMZ from a pure bacterial culture from said strain of interest, followed by DNA sequencing according to various well-known methods, for example Sanger, Roche 454, Illumina, Oxford Nanopore and then the sequenced genome is assembled by biocomputing and the sequences obtained are analyzed. Finally, the genomes of interest are compared two by two to calculate the ANI.

According to one variant, the invention also relates to a bacterial strain derived from Christensenella minuta, characterized in that it comprises a nucleotide sequence having at least 99.90% ANI identity with the nucleotide sequence of the bacterial strain DSM 33715.

Preferentially, said strain according to the invention comprises a nucleotide sequence having at least 99.91%, at least 99.92%, at least 99.93%, at least 99.94%, at least 99.95%, at least 99.96%, at least 99.97%, at least 99.98%, at least 99.99% ANI identity with the nucleotide sequence of the bacterial strain DSM 33715.

Such a strain is therefore a bacterial strain of Christensenella minuta derived from the bacterial strain deposited under the number DSM 33715 making it possible to maintain or improve the described capacities of the DSM 33715 strain in the context of the present invention.

Said derived strain can thus be produced naturally or intentionally by mutagenesis methods known in the prior art. By way of example, the mutagenesis methods that can be implemented within the scope of the present invention are the growth of the original microorganism in the presence of mutagenic or stress-producing agents, or by genetic engineering aiming to modify specific or non-specific genes such as directed mutagenesis or random mutagenesis. According to one object of the invention, the strain derived from the DSM 33715 strain of C. minuta is a genetically modified mutant.

According to a particularly preferred embodiment, the invention relates to the strain of Christensenella minuta filed under number DSM 33715 with Institut Leibniz DSMZ (Deutsche Sammlung von Mikroorganismen und Zellkulturen GmbH, or German Collection of Microorganisms and Cell Cultures GmbH). Said strain has a 16S rDNA sequence, the sequence SEQ ID NO:1.

Another subject matter of the invention relates to the culture supernatant obtained from the bacterial strain DSM 33715 and/or the derived strain. Preferentially, the culture supernatant comprises and at least one of the constituents selected from a bacterial cell compound, a bacterial cell debris, a metabolite and/or molecule(s) secreted by the strain and/or the derived strain, or combinations thereof.

Thus, the cell compounds and/or cell debris may be components of the wall, nucleic acids, membrane components, proteins, lipids, etc. The secreted metabolites or molecules may be any molecule produced or modified by the bacterium due to its metabolic activity during its growth, its use in technological processes (for example, but not limited thereto, methods for producing food or medicaments). By way of example, the metabolites or molecules may be proteins, amino acids, enzymes, lipids, nucleic acids, etc. “Metabolite and/or molecule(s) secreted by the strain and/or the derived strain” means a molecule produced and exported or released outside the bacterium by the bacterium.

Thus, the invention also relates to the culture supernatant obtained from the bacterial strain according to the invention.

### Composition

The strain(s) according to the invention or derived strain(s) and/or the culture supernatant described previously and the bacteria derived from said strains are advantageously administered in a composition.

Thus, the invention relates to a composition comprising: (i) at least one bacterial strain according to the invention and/or (ii) a supernatant according to the invention and (iii) at least one acceptable excipient.

In particular, the composition comprises at least one bacterial strain filed under number DSM 33715 and/or at least one strain having at least 99.5% identity with the 16S rDNA sequence of the DSM 33715 strain and/or the culture supernatant obtained from said strains and at least one acceptable excipient and/or a physiologically acceptable medium.

According to one embodiment, the composition may comprise the bacterial strain according to the invention in any form producing the expected effects and the efficiency described in the present application. In particular, the strain can be in living (cultivable or not), dead, semi-live, attenuated or inactivated form. The dead, semi-living, attenuated or inactivated form can be obtained by various techniques known to a person skilled in the art. Mention will be made, for example, of the following techniques: irradiation, thermal inactivation or freeze-drying, in particular by heat, exposure to an appropriate pH, UV, gamma rays, X-rays or high pressure.

The term “semi-living” refers to a bacterium with a low physiological activity whose ability to proliferate is reduced, temporarily or definitively. The term “inactivated” refers to a bacterium which is no longer capable, temporarily or definitively, of proliferating. The term “dead” refers to a bacterium that is definitively no longer capable of proliferating. The dead or inactivated bacteria can have intact or broken cell membranes. Thus, the term “inactivated” also refers to the obtained extracts and lysates of bacteria.

The composition according to the invention can also comprise at least one additional compound. An additional compound may in particular be an ingredient, a molecule, an active principle, a microorganism, a bacterium or a mixture of bacteria.

Preferentially, the additional compound is a microorganism different from the bacterial strain according to the invention or from the derived strain. More preferably, the microorganism is a bacterium of the genus Christensenella. As an example, mention may be made of a bacterium belonging to the species C. minuta, C. timonensis, C. massiliensis and mixtures thereof.

According to one variant, the additional compound is a probiotic and/or a prebiotic. The prebiotic may be, for example, at least one prebiotic selected from galactooligosaccharides, fructooligosaccharides, inulins, arabinoxylans, beta-glucans, lactoglobulins and/or beta-caseins.

According to another variant, the additional compound can be:
at least one bacterium producing lactic acid that makes it possible to create an anaerobic environment favorable to Christensenellaceae such that at least one bacterium selected from the bacteria of the genus Lactobacillus spp., Bifidobacterium spp., Streptococcus spp. and/or
at least one bacterium producing butyric acid that makes it possible to create an environment favorable to Christensenellaceae such as a bacterium of the genus Ruminococcaceae and Lachnospiraceae,
at least one other organism promoting the anaerobic conditions required for survival of the Christensenellaceae such that at least one yeast selected from among Saccharomyces spp. or microorganisms of the Metanobacteriaceae family, and/or
at least one bacterium associated with the Christensenellaceae ecosystem, since they facilitate their survival in the intestine, such as at least one bacterium chosen from bacteria of the phylum Firmicutes, Bacteroidetes, Actinobacteria, Tenericutes, and Verrucomicrobia, and/or
at least one polyphenol such as for example at least one polyphenol selected from quercetin, kaempferol, resveratrol, flavones (such as luteolin), flavan-3-ols or catechins, flavanones (such as naringinin), isoflavones, anthocyanidins, oligo-proanthocyanidins, and/or
at least one mineral and/or at least one vitamin and/or at least one nutritional agent, and/or
at least one pharmaceutical active ingredient preferentially selected from non-steroidal anti-inflammatory medicaments, antibodies directed against pro-inflammatory targets (such as anti-TNF-alpha or anti-IL6), anti-inflammatory targets, analgesics, antimicrobials, corticosteroids, anabolic steroids, antidiabetics, thyroid agents, antidiarrheals, cough suppressants, antiemetics, anti-ulcers, laxatives, anticoagulants, erythropoietin, immunoglobulins, immunosuppressors, growth hormones, hormonal medications, estrogen receptor modulators, alkylating agents, antimetabolites, mitotic inhibitors, radiopharmaceuticals, antidepressants, antipsychotics, anxiolytics, hypnotics, sympathomimetics, stimulants, donepezil, tacrine, medicaments for asthma, beta-agonists, inhaled steroids, leukotriene inhibitors, cromoglycates or cromoglycidic acids, epinephrine, dornase alpha, cytokines, cytokine antagonists, Janus Kinase inhibitors, immunomodulators, lipid-lowering medicaments, cholesterol-lowering medicaments, antihypertension medicaments.

When the additional compound is a microorganism different from the strain according to the invention, the microorganism is preferentially selected from Akkennansia muciniphila, Faecalibacterium prausnitzii, Anaerobutiryricum hallif, Hafnia alvei, Roseburia intestinalis, Rosaburia hominis, Roseburia faecis, Roseburia inuhinivorans, Dysosmobacter welbionis, Oscillospira guillennondii, Lactiplantibacillus plantarum, Lacticaseibacillus casei, Latilactobacillus sakei, Ligilactobacillus salivarius, Limosilactobacillus fermentum, Limosilactobacilus reuteri, Levilactobacillus brevis, Bifidobacterium pseudolongum, Bifidobacterium longum, Bifidobacterium infantis, Bifidobacterium animalis, Bifidobacterium adolescentis, Bifidobacterium biffdum, Bifidobacterium breve, Bifidobacterium lactis, Bifidobacterium stercoris and Bifidobacterium thermophilum.

When the additional compound is a pharmaceutical active ingredient, it is preferentially an active ingredient selected from 5-ASA, mesalamine, olsalazine, budesonide, tofacitinib, filgotinib, upadacitinib, infliximab, adalimumab, golimumab, certolizumab, vedolizumab and ustekinumab.

Preferentially, the composition according to the invention is in any form acceptable to be administered to a subject, preferentially a human or non-human animal. Thus, the composition according to the invention also relates to veterinary uses.

Preferably, the composition according to the invention is in solid, liquid or lyophilized form.

When the composition is in liquid form, it may in particular comprise bacterial strains according to the invention and/or of the derived strain and/or a culture medium physiological acceptable to said bacteria which makes it possible to maintain them, such as for example preferentially the anaerobic Columbia agar medium enriched with sheep blood, or an equivalent medium not containing any derived product of animal origin.

When the composition according to the invention is in solid form, the bacterial strains according to the invention and/or of the derived strain can be present in lyophilized form, and may also comprise excipients such as, for example, microcrystalline cellulose, lactose, sucrose, fructose, levulose, starches, stachyose, raffinose, amylum, calcium lactate, magnesium sulfate, sodium citrate, calcium stearate, polyvinylpyrrolidone, maltodextrin, galactooligosaccharides, fructooligosaccharides, pectins, beta-glucans, lactoglobulins, isomaltooligosaccharides, polydextroses, mannitol, sorbitol and/or glycerol.

According to another preferred embodiment, the composition according to the invention is in a form suitable for oral, nasal, parenteral, rectal, sublingual, ocular, auricular, intramuscular, intravenous, inhaled or cutaneous administration.

The composition may be in any suitable form. Thus, the composition according to the invention may be in a form selected from a powder, microencapsulated powder, capsule, tablet, pellet, granule, emulsion, suspension, suppository, inhaler, and syrup.

According to a particularly suitable embodiment, the composition according to the invention may be in gastro-resistant form, for example a coated tablet containing microencapsulated bacteria. Said composition can thus be provided with a gastro-resistant coating or a coating resistant to gastric juice, in order to ensure that the bacterium (bacteria) comprised in said composition can pass through the stomach. The release of the bacterium (bacteria) can thus occur for the first time in the upper intestinal tract.

In particularly preferred fashion, the composition comprises 10⁴ to 10¹² colony forming units (CFU) of bacteria per daily dose of composition to be administered, preferentially 10⁶ to 10¹² colony forming units (CFU) of bacteria per daily dose of composition to be administered.

Preferentially, this corresponds to a daily dose of bacteria to be administered, regardless of the weight of the person or of the animal. Preferably, this dose is administered in a single time. More preferentially, the useful composition comprises 10⁵ to 10¹¹ CFU or 10⁷ to 10¹¹ of bacteria per daily dose to be administered, even more preferentially 10⁹ CFU.

Said bacteria are a mixture of bacteria corresponding to or derived from the bacterial strain DSM 33715 and/or corresponding to the derived strain.

The term CFU (Colony Forming Unit) is a unit making it possible to count the number of bacteria capable of giving rise to a colony during propagation, that is to say viable bacteria. It should be understood that non-viable bacteria can also be present in the compositions and that in general they should not have a negative effect on the properties of the living bacteria of the composition, and, on the contrary, they can exert an effect on themselves.

Preferentially, the daily dose is measured per gram or milliliter of the final composition according to the invention.

According to another preferred object, when the composition according to the invention comprises a mixture of bacteria, said mixture comprising at least living bacteria, the composition preferentially comprises at least 1% of living bacteria (in number), more preferentially at least 10% living bacteria (in number), even more preferentially at least 50% living bacteria (in number).

The living bacteria are preferentially a mixture of bacteria corresponding to the bacterial strain DSM 33715 and/or corresponding to the derived strain having at least 99.90% ANI identity with the nucleotide sequence of the bacterial strain DSM 33715.

In the case of the implementation of a supernatant of any one of the aforementioned bacterial strains, the composition according to the invention including said supernatant may in particular comprise a content thereof of between 0.1 and 99% by weight, namely from 5 to 95% by weight, in particular from 10 to 90% by weight and more particularly from 15 to 85% by weight, relative to the total weight of the composition.

Thus, a composition according to the invention may comprise a supernatant content of between 0.1 and 99% by weight, namely from 5 to 95%, in particular from 10 to 90% by weight, more particularly from 15 to 85% by weight, relative to the total weight of the composition.

When the composition is intended for therapeutic use, the composition may comprise at least one pharmaceutically acceptable excipient.

When the composition comprises such a pharmaceutically acceptable excipient, the composition according to the invention is preferentially a pharmaceutical composition.

The invention thus relates to a pharmaceutical composition comprising the ingredient(s) included in the composition according to the invention and according to one of any embodiments described above.

Said pharmaceutical composition according to the invention has at least one application in the improvement of the physical, physiological or psychological well-being of a subject with a disease, which results in an improvement in the general state of health of said subject or a reduction in the risk of disease. Said pharmaceutical composition may be a medicament.

According to another aspect, the composition according to the invention may be in the form of a food product, a beverage, a nutraceutical, a food additive, a food supplement or a dairy product.

When the composition according to the invention is comprised in a food supplement for oral administration, the latter may be in the form capsules, gel capsules, soft capsules, tablets, sugar-coated tablets, pills, pastes, pellets, gums, drinkable solutions or emulsions, a syrup or a gel.

A food supplement according to the present invention may further comprise a sweetener, a stabilizer, an antioxidant, an additive, a flavoring agent and/or a colorant. The formulation of the latter is carried out by means of the usual methods for producing sugar-coated tablets, gel capsules, gels, hydrogels for controlled release, emulsions, tablets or capsules.

A composition according to the present invention may also be in the form of a nutritional composition. Such nutritional composition according to the present invention may be in the form of a yogurt, a cereal bar, breakfast cereals, a dessert, frozen food, a soup, a pet food, a liquid suspension, a powder, a tablet, a gum or a sweet.

A nutritional composition according to the present invention may further comprise at least one ingredient selected from: antioxidants, fish oils, DHA, EPA, vitamins, minerals, phytonutrients, protein, lipid, probiotics, prebiotics and combinations thereof.

### Use

The invention therefore relates to the bacterial strain according to the invention and/or the derived strain, the supernatant according to the invention, more particularly the composition according to the invention for its use as a medicament.

In particular, the invention makes it possible to provide an effective amount of bacterial strains according to the invention or of the derived strain, to be used as a medicament.

The use as a medicament in the context of the present invention refers to human or veterinary use, said medicament is then administered to a human subject or a non-human animal subject. Human subject is also understood to mean an adult or child or a baby, including a newborn or an infant.

Preferentially, the composition according to the invention is intended to be used in the prevention and/or treatment of dysbiosis of the microbiota, in particular intestinal microbiota and/or diseases associated with dysbiosis of the intestinal microbiota and/or chronic diseases and/or obesity and/or metabolic diseases and/or inflammatory diseases and/or cancers.

More preferably, the composition according to the invention is intended to be used in the prevention and/or treatment of chronic inflammatory bowel diseases, in particular of a disease chosen from Crohn's disease, ulcerative colitis, hemorrhagic ulcerative rectocolitis, diverticulitis, esophagitis, gastritis, pancreatitis, peptic ulcers, irritable bowel syndrome.

According to one variant, the invention also relates to the composition according to the invention for its use as a medicament, in the prevention and/or treatment of at least one disease chosen from:
Metabolic diseases, selected from non-insulin-dependent diabetes, gestational diabetes, NASH, hepatic steatosis, pancreatic steatosis, hyperlipidemia, hypercholesterolemia, infertility linked to excess weight, urinary incontinence linked to excess weight,
Other chronic metabolic diseases, including thyroiditis,
cardiac and vascular diseases, selected from atherosclerosis, thrombopathies, acute pericarditis and chronic constrictive pericarditis, arterial hypertension, vasculitides
liver and bile duct diseases, selected from hepatitis, primary biliary cirrhosis, primary sclerosing cholangitis, cirrhosis, hepatic encephalopathy, vesicular lithiasis
joint diseases linked to excess weight, selected from osteopenia, osteoporosis, osteoarthritis, vertebral disc inflammation
neurodegenerative diseases, selected from Alzheimer's disease, Parkinson's disease, motor neuron diseases such as amyotrophic lateral sclerosis, primitive lateral sclerosis and Kennedy disease
cancers linked to metabolism and/or to dysbiosis of the microbiota, selected from hepatocarcinomas, gastrointestinal tract cancers such as esophageal, stomach and colorectal cancer, pancreatic carcinoma, neuroendocrine tumors (NETs) of the gastrointestinal-pancreatic system, hepatic tumors, gallbladder and bile duct tumors, renal tumors, glioblastoma, lymphoma, multiple myeloma, chronic myeloid lukemia, chronic myeloproliferative diseases, lung carcinoma
auto-immune diseases, selected from insulin-dependent diabetes, rheumatoid arthritis, ankylosing spondylitis, psoriatic arthritis, systemic lupus erythematosus, polyendocrine autoimmune syndrome
atopic skin diseases, selected from eczema, psoriasis
chronic inflammatory bowel diseases, selected from Crohn's disease, hemorrhagic ulcerative rectocolitis, diverticulitis, esophagitis, gastritis, pancreatitis, gastrointestinal ulcers, irritable bowel syndrome
respiratory function disorders, selected from asthma, cystic fibrosis, chronic obstructive pulmonary disease, interstitial lung diseases and pulmonary fibroses, sleep apnea syndrome (OSAS)
pneumonias, selected from infectious pneumonia, influenza pneumonia and avian flu, severe acute respiratory syndrome (SARS), pneumocystis pneumonia
Infectious diarrhea, selected from Clostridium difficile infection, EHEC infection, salmonella gastroenteritis, campylobacter enteritis, food poisoning by enterotoxin-producing bacteria, cholera, yersinosis, shigellosis, cryptosporidiosis, listeriosis
Food allergies, selected from celiac disease, lactose intolerance, bile salt malabsorption syndrome
Inflammatory kidney disorders or others related to dysbiosis of the microbiota, selected from urethritis, chronic renal failure, urolithiasis
Other inflammatory disorders, selected from multiple sclerosis, lymphangitis
Neurological diseases related to dysbiosis of the microbiota, selected from anorexia, bulimia, depression, bipolar syndrome, autism, schizophrenia, Tourette syndrome.

According to a final subject matter, the invention also relates to a non-therapeutic use of the composition according to the invention for maintaining and/or reinforcing the intestinal microbiota and/or supporting the diversity of the intestinal microbiota and/or promoting the increase of beneficial bacteria in the intestinal microbiota and/or promoting the loss of weight in a healthy subject.

Preferentially, when the composition is intended to reinforce the intestinal microbiota and/or support the diversity of the intestinal microbiota and/or promote the increase of beneficial bacteria in the intestinal microbiota and/or promote weight loss in a healthy subject, then the composition is in the form of a food product, a beverage, a nutraceutical, a food additive, a food supplement or a dairy product.

The invention is now shown by examples of bacteria useful according to the invention, methods of cultures of these bacteria, examples of compositions containing them and results of tests demonstrating their efficacy, which are presented by way of illustration only.

### EXAMPLES

### Example 1: Characterization of C. minuta According to the Invention Deposited Under the Number DSM 33715

The bacterial strain C. minuta DSM 33715 can be characterized by the following method.

The native bacteria of the DSM 33715 strain (10 μL of culture medium assayed at 10⁹ CFU/mL) were deposited on grids made of ionized carbon (Delta Microscopy, Toulouse, France) and a negative coloring was carried out with Nano-Tungsten (Nano-W, Nanoprobes, LFG Distribution, France).

The observation of the bacteria mounted on the grid was carried out with a transmission electron microscope (Talos F200S G2—Thermofisher-Eindhoven) at 200 kV, equipped with a 4K*4K One View camera (Gatan, Paris, France). The preparation of the samples, thus the acquisitions, were carried out at the Bordeaux Imaging Center (member of France-Biolmaging, ANR-10-INBS-04).

Oxidase activity was determined through the use of oxidase test strips (Sigma-Aldrich) by evaluating the color change after adding a liquid bacterial culture drop.

The catalase activity was determined at two concentrations of H₂O₂ (3 and 15%), added to a colony of DSM 33715, the catalase status being visually evaluated by the formation of bubbles.

Gram staining was carried out following the traditionally procedure.

The tolerance to bile and to pH were evaluated in a liquid modified GAM medium (HyServe). For the tolerance to bile, media containing 0-2-4-6 and 8% Oxgall (Difco Laboratories) were used, corresponding respectively to 0-20-40-60 and 80% bile. For pH tolerance, media at pH 4-5-6-7 and 8 were used. All the media were degassed in the anaerobic chamber before inoculation with 10 μl of a bacterial suspension at 0.5 of McFarland. The optical density at 600 nm was taken at 0 h, 24 h, 48 h, 72 h and 96 h.

The antibioresistance tests were carried out through the use of minimum inhibitory concentration (MIC) according to the dilution reference method in the CLSI (Clinical and laboratory Standards Institute) agar medium. The stock solutions of each antibiotic were prepared and sterilized by filtration at 0.2 μ: ampicillin (Sigma-Aldrich), tetracycline (Sigma-Aldrich, Chloramphenicol (Sigma-Aldrich), clindamycin (Sigma-Aldrich), meropenem (USP), piperacillin (USP), tazobactam (USP), and ceftriaxone (USP). The Brucella agar medium (BD BBL) supplemented with 5 μg/ml of hemin (Sigma-Aldrich), 1 μg/ml of vitamin K1 (Sigma-Aldrich) and 5% (v/v) of hemolyzed sheep blood (laked) (Hemostat) was used as a test medium. The agar dishes with different dilutions of antibiotic were prepared and degassed in the anaerobic chamber before inoculation. The inoculum of DSM 33715 was resuspended in the standard 0.5 of McFarland in peptone water. 2 μl of the inoculum were deposited on the surface of the agar (each spot corresponding approximately to 10⁵ CFU/ml). A triplicate is carried out by concentration of antibiotics. The dishes were then incubated for 6 days at 37° C. under anaerobic conditions. The MIC values were determined according to the CLSI Guidelines and were interpreted as sensitive, intermediate or resistant, based on the CLSI breakpoints for anaerobes.

### Characterization Results

The DSM 33715 strain is strictly anaerobic with an exponential growth phase between 20 and 40 h and a stationary phase beginning at 40 h, non-motile, non-sporulating, negative for oxidase and catalase activity and is gram negative. DSM 33715 is negative for the majority of the enzymes tested, with the exception of the acidification of glucose, xylose, rhamnose and arabinose, which is consistent with the description of Christensenella minuta made by Morotomi et al. It has the following enzyme activities: 0-Glucosidase, 0-Arabinosidase, Glutamic Acid Decarboxylase, as well as Esterase, Acid Phosphatase and Napthol-AS-Bl-Phosphatase. Resistance to biliary acids was observed up to 80 g/l of Oxgall, corresponding to 80% bile. DSM 33715 also grows in media with pH ranging from 6 to 9. Finally, the DSM 33715 strain shows resistance to ampicillin and tetracycline.

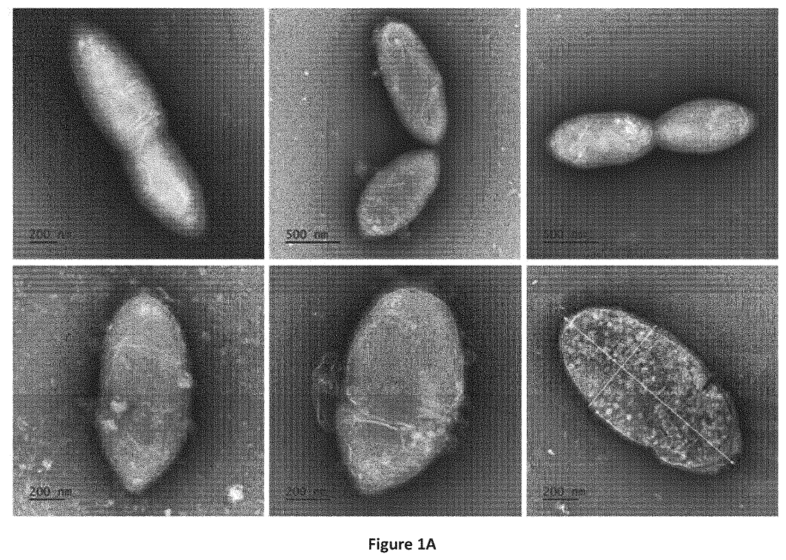

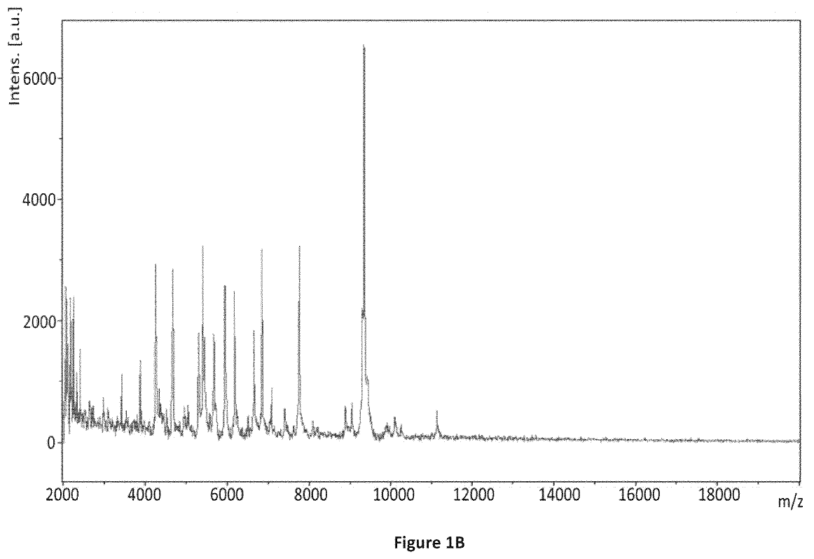

FIG. 1A shows the image of a Christensenella minuta bacterium according to the invention produced by transmission electron microscopy in negative coloring (magnification: 36kX). The average dimensions of a bacterium of the DSM 33715 strain are: length=1.27+−0.28 mm; width=0.507+−0.04 mm; with a membrane thickness=29.4 nm (mean obtained from 41 bacteria). The strain has a short shape with tapered ends, appearing in singles, pairs, or rosettes.

The strain according to the invention can also be characterized by the analysis of a MALDI spectrum.

Thus, a pure culture of the DSM 33715 strain is incubated in an agar medium and subjected to a matrix-assisted laser desorption/ionization (MALDI) analysis using a MALDI Biotyper instrument (Bruker) and using the standard method recommended by the manufacturer.

MALDI identification is a known method for identifying a bacterial colony by genus and species. This is characteristic of the molecular composition of the surface of said bacterium. Thus, FIG. 1B represents the MALDI spectrum characteristic of a pure culture of Christensenella minuta according to the invention (DSM 33715). This makes it possible to confirm that said strain belongs to the species C. minuta, thus validating the purity of the culture.

### Example 2: Comparison Between the Strain According to the Invention (DSM 33715) and Strains of C. minuta from the Prior Art

The inventors also compared the strain according to the invention (DSM 33715) with known strains of C. minuta such as the reference strain deposited under number DSM 22607, the strain deposited under number DSM 32891 and the strain deposited under number DSM 33407 according to several parameters such as the sequence of the 16S rDNA, the size of the base pair genome (bp), the full genome phylogenetic distance (ANI), the coding sequence number (CDS) and examples of genes specific to the strain.

The results are presented in tables 1 to 3 below.

TABLE 1

| | | Size of | | CDS (Coding | Example of |
|---|---|---|---|---|---|
| | | genome | ANI (genome | Sequence | specific |
| | 16S rDNA | (bp) | homology %) | number) | genes |
| DSM22607 | SEQID NO: 1 | 2944440 | 99.84% | 2765 | nd |
| DSM 33715 | SEQID NO: 1 | 2959549 | | 2773 | nd |

TABLE 2

| | | Size of | | CDS (Coding | Example of |
|---|---|---|---|---|---|
| | | genome | ANI (genome | Sequence | specific |
| | 16S rDNA | (bp) | homology %) | number) | genes |
| DSM 32891 | SEQID NO: 1 | 2841506 | 99.37% | 2663 | gene |
| | | | | | encoding |
| | | | | | RibZ |
| DSM 33715 | SEQID NO: 1 | 2959549 | | 2773 | gene |
| | | | | | encoding a |
| | | | | | beta- |
| | | | | | mannanase |

TABLE 3

| | | Size of | | CDS (Coding | Example of |
|---|---|---|---|---|---|
| | | genome | ANI (genome | Sequence | specific |
| | 16S rDNA | (bp) | homology %) | number) | genes |
| DSM 33407 | SEQID NO: 1 | 2791808 | 99.69% | 2595 | gene |
| | | | | | encoding |
| | | | | | PknD |
| DSM 33715 | SEQID NO: 1 | 2959549 | | 2773 | Gene |
| | | | | | encoding |
| | | | | | TrxB |

Thus, the strain according to the invention (DSM 33715) has the same 16S rDNA sequence as the other three already-known strains of C. minuta despite having different metabolic properties and anti-inflammatory potential. However, the size of the genome, the whole genome, the number of coding sequences, and certain genes specific to said strain make it possible to characterize and differentiate the strains from one another. In particular, the strain according to the invention has the largest genome and a greater number of coding sequences than the already-known strains.

### Example 2: Anti-Inflamatory Effect of the Strain According to the Invention

In the present study, the inventors studied the immunomodulatory pro- and anti-inflammatory effect of the DSM 33715 strain.

The study protocol is as follows.

PBMCs from 3 healthy donors (Lonza) were inoculated into 24-well plates at 1×10⁶ cells/wells in RPMI medium (Gibco) supplemented with 2% fetal bovine serum (FCS, Gibco) and incubated at 37° C./5% C02 for 24h in the presence of the bacterium DSM 33715 at a MOI of 50 or bacterial-Glycerol medium as control. After 24 h, the cell culture supernatants were collected and stored at −80° C. until the NL-10 was quantified by ELISA. The IL-10 assay was carried out by BioLegend specific ELISA, according to the manufacturer's instructions. The absorbance at 460 nm was read using the FluoStar Omega microplate reader of BMG Labtech.

The THP-1 (ECACC) cells were seeded at 5×10⁵ cells/wells in 24-well plates in RPMI medium (Gibco) supplemented with 10% FCS (Gibco) in the presence of 100 ng/ml of PMA (Enzo Life Sciences) for 48 h, in order to differentiate the monocytic cells into macrophages of the MO type. The cells were then washed with PBS-1× (Gibco) and cultivated for an additional 24 h in RPMI medium with 2% FCS. The cells were then stimulated for 24 h with the DSM 33715 bacterium at a MOI of 50 or PBSIX-Glycerol as control. After 24 h, the cell culture supernatants were collected and stored at −80° C. until the IL-10 was quantified by ELISA. The IL-10 assay was carried out by BioLegend specific ELISA, according to the manufacturer's instructions. The absorbance at 460 nm was read using the FluoStar Omega microplate reader of BMG Labtech.

Statistics: One-way ANOVA followed by Dunett's multiple comparisons, * p<0.05, ** p<0.01, *** p<0.001, **** p<0.0001.

### Results

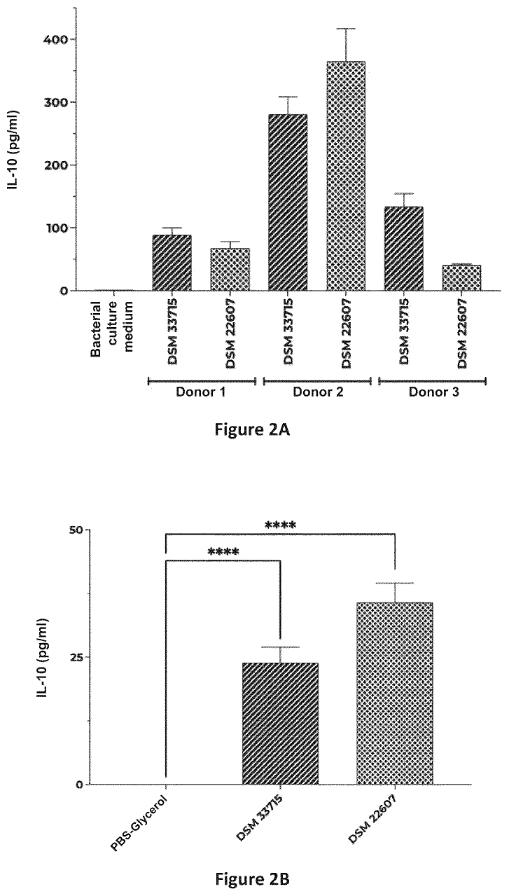

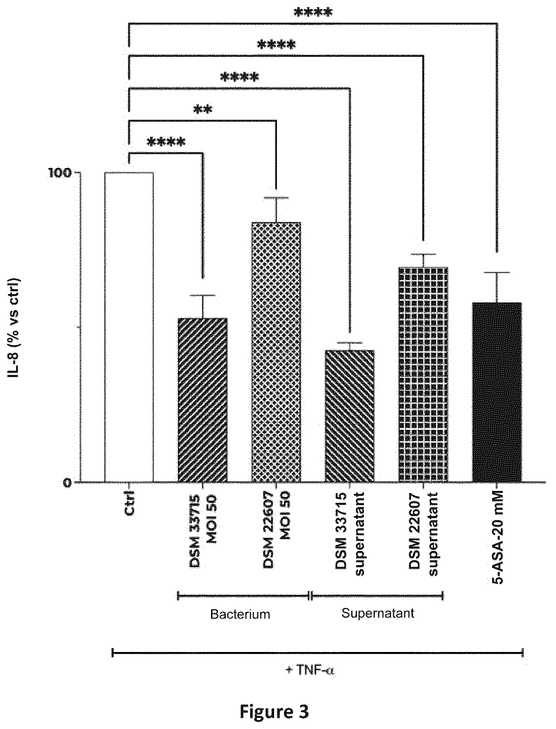

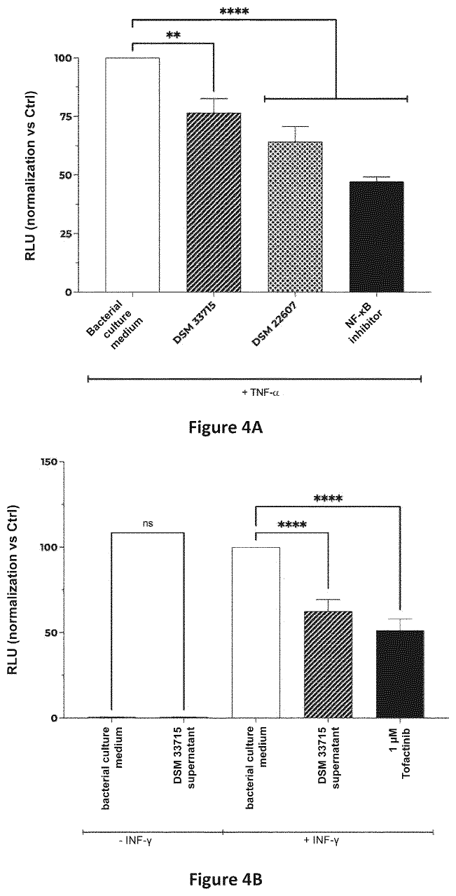

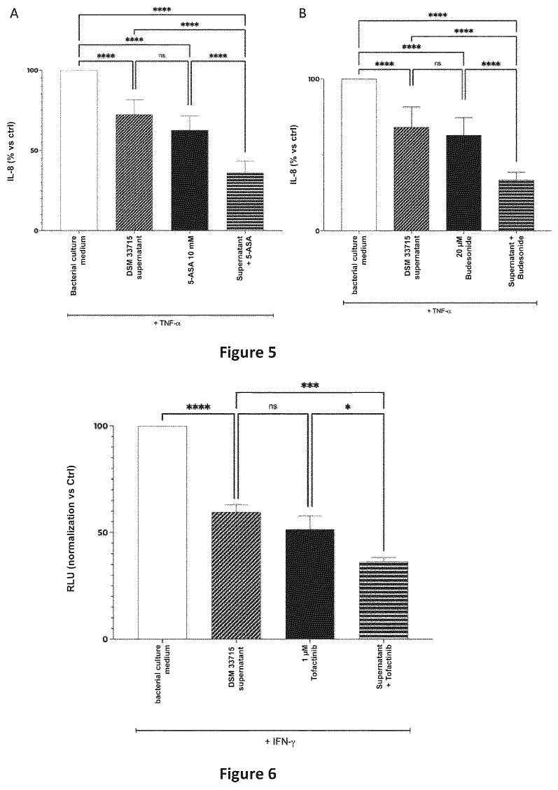

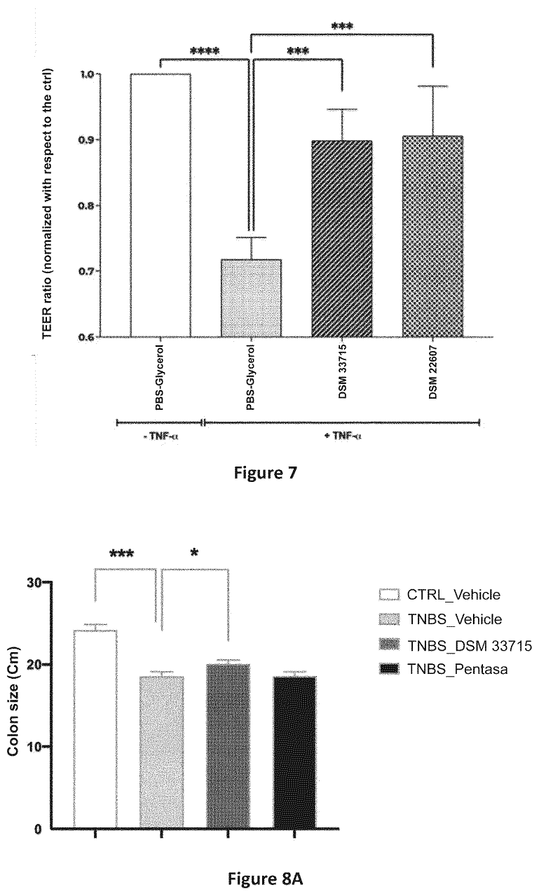

In order to evaluate the immunomodulatory effect of the DSM 33715 strain, the PBMCs cells were incubated for 24 h in the presence of the DSM 33715 bacterium at a MOI of 50. The chemokine IL-10 is an anti-inflammatory cytokine. The values represent the secretion of IL-10 induced by the strain according to the invention (DSM 33715). For 2 of the 3 donors, DSM 33715 induces greater IL-10 production than a reference strain of C. minuta DSM 22607 (FIG. 2A).

The fact that the production of IL-10 production, an anti-inflammatory cytokine, was induced by DSM 33715 was also demonstrated by the THP-1 cell model differentiated into MC-type macrophages by treatment with PMA (FIG. 2B).

The DSM 33715 strain thus has an anti-inflammatory immuno-modulating effect that is greater than the reference strain of C. minuta and therefore has a high therapeutic potential for inflammatory diseases.

### Example 3: Anti-Inflammatory Effect of the Strain According to the Invention

In the present study, the inventors studied the anti-inflammatory effect of the DSM 33715 strain on an HT-29-producing adenocarcinoma line of IL-8 after stimulation with TNF-α.

The HT-29 cells (ECACC) were inoculated into 24-well plates at 3×10⁵ cells per well in McCoy's 5A (Gibco) medium supplemented with 10% fetal calf serum (FCS, Gibco) and incubated at 37° C./5% C02 for 24 h. The cells were washed with PBS-1× (Gibco) and cultivated for an additional 24 h in McCoy's 5A medium at 2% FCS. The cells were then stimulated for 6 hours with 5 ng/ml of TNF-α in the presence of 50 bacteria per 1 HT-29 cell (MOI 50) of DSM 33715, or 10% of supernatant or PBSIX-Glycerol or bacterial culture medium as control (ctrl). DSM 22607 is the reference strain. 5-ASA (20 mM) is an anti-inflammatory agent used in the control. After 6 h, the cell culture supernatants were collected and stored at −80C until the IL-8 was quantified by ELISA. The IL-8 assay was carried out by BioLegend specific ELISA, according to the manufacturer's instructions. The absorbance at 460 nm was read using the FluoStar Omega microplate reader of BMG Labtech. 6 independent experiments were carried out, in duplicate. Statistics: one-way ANOVA followed by Dunett's multiple comparisons, * p<005, ** p<0.01, *** p<0.001, **** p<0.0001.

### Results

To evaluate the immunomodulatory effect of the DSM 33715 strain, the HT-29 cells were stimulated with the pro-inflammatory cytokine TNF-α and the bacterium DSM 33715 at a MOI of 50 at the start of the stationary growth phase or its supernatant, for 6H. Chemokine IL-8 is secreted by HT-29 in an inflammatory state induced by TNF-α (Ctrl). The values represent the mean normalized as a percentage relative to the control+TNF-α, (PBS-Glycerol for the bacterium and the bacterial culture medium for the supernatant), representing the secretion of IL-8 induced by stimulation with TNF-α, that is 100%, In the presence of DSM 33715 or of its supernatant, the IL-8 secretion is reduced by 50% relative to the control (p<0.0001, Dunnett's multiple comparisons test). Thus, the strain DSM 33715 and its supernatant have an anti-inflammatory immunomodulator effect.

### Example 4: Anti-Inflammatory Effect of the Strain According to the Invention

In the present study, the inventors studied the anti-inflammatory effect of the DSM 33715 strain on the NF-0B and JAK/STAT inflammation signaling pathways.

HT-29 cells at a density of 3×10⁵ cells/well, in 24-well plate, were transfected according to a reverse-transfection protocol, with 200 ng of pRelA-luc vector (NF-0B signaling pathway) or pGL4 luc2P GAS-RE (IFN-γ-dependent JAK/STAT signaling pathway) and 10 ng of pRL-TK vector (Promega), using X-tremeGENE HP DNA transfection reagent with a 3:1 ratio (Roche). To summarize, the reactive/DNA transfection complexes were prepared in a final volume of 50 μl of McCoy's 5A without serum and without antibiotic, the appropriate amount of each plasmid was added and homogenized, and then the transfection agent was added (3:1). The transfection complexes were carefully mixed and incubated at room temperature for 15 minutes. After incubation, the transfection complexes were added to the freshly resuspended HT-29 cells at the appropriate density in McCoy's 5A medium supplemented with 10% FCS, and then the cell/transfection complex mixtures were gently homogenized and placed on plates. The plates were incubated for 24 h in a humid atmosphere at 5% C02 and the medium was then replaced with medium at 2% FCS for 12 h, after washing with PBS IX. After 12 h of serum deprivation, the medium was removed and replaced with 2% FCS medium+/−5 ng/ml of TNF-α (NF-0B reporter system) or 100 μg/mi of IFN-γ (JAK/STAT reporter system) and 10% of supernatant of DSM 33715 or of bacterial culture medium as control or specific inhibitor of the signaling pathway (5 μM BAY11-7082 for the NF-8B pathway and 1 μM Tofacitinib for the JAK/STAT pathway). After 6 h of co-incubation, the cells were washed with PBS IX and lysed in 50 μl of passive lysis buffer (Promega) for 15 min at room temperature with orbital stirring, the protein lysates were then transferred into microtubes. A reporter Luciferase test (Dual-Luciferase reporter assay, Promega) was carried out with minor modifications to the manufacturer's instructions. To summarize, 2×20 μl of lysate were transferred into a 96-well white plate, and 50 μl reagent LAR II was added. The activity of the luciferase Firefly was read. Then 50 μl reagent Stop&Glo was added and the measurement of the Renilla activity was read with the FLUOstar Omega plate reader (BMG Labtech). The results were reported as the Firefly/Renilla ratio. Statistics: Sidak's multiple comparisons test. * p<0.05, ** p<0.01, *** p<0.001, **** p<0.0001

### Results

The DSM 33715 supernatant leads to a 25% decrease in the TNF-α-induced activation of the NF-0B pathway and a 40% decrease in the IFN-γ-induced activation of the JAK/STAT pathway. However, these pathways are particularly relevant since they are hyperactive in patients suffering from chronic inflammation, as in Crohn's disease and certain cancers. The NF-κB pathway is involved in cancers of epithelial origin, such as breast cancer; in gastrointestinal cancers, such as colorectal cancer. The Jak/STAT pathway is overactivated mainly in solid cancers, such as breast, lung, liver cancer, head, neck and stomach cancers.

### Example 5: Anti-Inflammatory Effect of the Strain According to the Invention in Combination with an Anti-Inflammatory Agent

In the present study, the inventors studied the anti-inflammatory effect of the DSM 33715 strain in combination with 5-ASA or Budesonide.

The HT-29 cells (ECACC) were inoculated into 24-well plates at 3×10⁵ cells per well in McCoy's 5A (Gibco) medium supplemented with 10% fetal calf serum (FCS, Gibco) and incubated at 37° C./5% C2 for 24 h. The cells were washed with PBS-1× (Gibco) and cultivated for an additional 24 h in McCoy's 5A medium at 2% FCS. The cells were then stimulated for 6 hours with 5 ng/ml of TNF-α in the presence of 10% of supernatant in stationary phase of DSM 33715 or of bacterial culture medium as control (ctrl), whether or not in the presence of anti-inflammatory agents. 5-ASA (10 mM) and budesonide (20 μM) are molecules used in the treatment of inflammatory diseases, such as ulcerative colitis and Crohn's disease. After 6 h, the cell culture supernatants were collected and stored at −80° C. until the IL-S was quantified by ELISA. The IL-8 assay was carried out by BioLegend specific ELISA, according to the manufacturer's instructions. The absorbance at 460 nm was read using the FluoStar Omega microplate reader of BMG Labtech. Statistics: Sidak's multiple comparisons test. * p<0.05, ** p<0.01, *** p<0001, **** p<0.0001.

### Results

In order to evaluate the immunomodulatory effect of the DSM 33715 strain in combination with an anti-inflammatory agent used in therapy, the HT-29 cells were stimulated with the pro-inflammatory cytokine TNF-α and 10% of the supernatant of DSM 33715 in the presence or absence of 5-ASA or Budesonide, for 6H. Chemokine IL-8 is secreted by HT-29 in an inflammatory state induced by TNF-α (Ctrl). The values represent the average normalized as a percentage relative to the control+TNF-α, representing the secretion of IL-8 induced by stimulation with TNF-α, that is 100%.

In the presence of DSM 33715, the secretion of IL-8 is reduced by 28% relative to the control, and by 38% in the presence of 5-ASA. The combination of the supernatant and 5-ASA leads to a 64% decrease (Panel A). There is therefore an improved effect of the supernatant and of the 5-ASA in the modulation of inflammation.

In the presence of DSM 33715, the secretion of IL-8 is reduced by 32% relative to the control, and by 38% in the presence of Budesonide. The combination of the supernatant and budesonide leads to a 67% decrease (Panel B). There is therefore an improved effect of the supernatant and budesonide in the modulation of TNF-α-induced inflammation.

### Example 6: Anti-Inflammatory Effect of the Strain According to the Invention in Combination with a JAK Inhibitor

In the present study, the inventors have studied the anti-inflammatory effect of DSM 33715 associated or not with Tofacitinib on the modulation of the JAK/STAT inflammation signaling pathway induced by IFN-γ.

HT-29 cells at a density of 3×10⁵ cells/well, in 24-well plate, were transfected according to a reverse-transfection protocol, with 200 ng of pGL4 luc2P GAS-RE (IFN-γ-dependent JAK/STAT signaling pathway) and 10 ng of pRL-TK vector (Promega), using X-tremeGENE HP DNA transfection reagent with a 3:1 ratio (Roche). To summarize, the reactive/DNA transfection complexes were prepared in a final volume of 50 μl of McCoy's 5A without serum and without antibiotic, the appropriate amount of each plasmid was added and homogenized, and then the transfection agent was added (3:1). The transfection complexes were carefully mixed and incubated at room temperature for 15 minutes. After incubation, the transfection complexes were added to the freshly resuspended HT-29 cells at the appropriate density in McCoy's 5A medium supplemented with 10% FCS, and then the cell/transfection complex mixtures were gently homogenized and placed on plates. The plates were incubated for 24 h in a humid atmosphere at 5% C02 and the medium was then replaced with medium at 2% FCS for 12 h, after washing with PBS IX. After 12 h of serum deprivation, the medium was removed and replaced with 2% FCS medium+/−100 μg/ml of IFN-γ (JAK/STAT reporter system) and 10% of supernatant of DSM 33715 or of bacterial culture medium as control or specific inhibitor of the signaling pathway (5 μM BAY11-7082 for the NF-0B pathway and 1 μM Tofacitinib for the JAKISTAT pathway). After 6 h of co-incubation, the cells were washed with PBS IX and lysed in 50 μl of passive lysis buffer (Promega) for 15 min at room temperature with orbital stirring, the protein lysates were then transferred into microtubes. A reporter Luciferase test (Dual-Luciferase reporter assay, Promega) was carried out with minor modifications to the manufacturer's instructions. To summarize, 2×20 μl of lysate were transferred into a 96-well white plate, and 50 μl reagent LAR II was added.

The activity of the luciferase Firefly was read. Then 50 μl reagent Stop&Glo was added and the measurement of the Renilla activity was read with the FLUOstar Omega plate reader (BMG Labtech). The results were reported as the Firefly/Renilla ratio. Statistics: Sidak's multiple comparisons test. * p<0.05, ** p<0.01,*** p<0.001, **** p<0.0001

### Results

The DSM 33715 supernatant leads to a 40% decrease in the IFN-γ-induced activation of the JAKISTAT pathway, Tofacitinib leads to a 49% reduction, and the combination of the supernatant with Tofacitinib leads to a 64% decrease in the IFN-γ-induced activation of the JAK/STAT pathway. There is therefore an improved effect of the supernatant and Tofacitinib in the modulation of IFN-γ-mediated inflammation.

### Example 7: Effect of the Strain According to the Invention in Protecting the Intestinal Epithelium

In the present study, the inventors studied the effect of the DSM 33715 strain on the integrity of the intestinal barrier by measuring transepithelial resistance.

The Caco-2 (ECACC) cells were seeded at 2×10⁴ cells per well in 24-well plates containing inserts with a permeable membrane made of Transwell polyester (Corning Life Science). The cells were cultivated in DMEM medium supplemented with 20% fetal calf serum (FCS, Gibco) and incubated at 37° C./5% C02. The transepithelial electrical resistance was verified every day from the seventh day of culture using an EVOM3 ohmmeter (WPI). The medium was replaced every 2 days for 8 to 10 days, until the optimal TER was reached (2000 Ω·cm²). When the reference TEER was reached, the cells were treated in the apical compartment with DSM 33715 or DSM 22607 at a MOI of 50 or PBS IX/Glycerol 10% (control) for 3 h, then 100 ng/ml of TNF-α was added or not in the basal compartment. The TEER was measured just before the addition of TNF-α (T0) and 6h (T6H) after the addition of TNF-α.

### Results

To evaluate the capacity of the DSM 33715 strain to restore or reinforce the intestinal barrier, the Caco-2 cells in polarized monolayer were pre-treated with DSM 33715, then sensitized with TNF-α. The TEER was compared to T0 (before addition of TNF) and T6H. The values represent the mean normalized relative to the control—TNF-α, representing the basal TEER. As expected, the treatment of Caco-2 with TNF-α induces an increase in permeability (Ctri+TNF-α). The DSM33715 strain is capable of restoring the epithelial barrier, with a return to the basal level.

### Example 8: Effect of the Strain According to the Invention in an In Vivo Model of Colitis

In the present study, the inventors studied the in vivo effect of TNBS-induced colitis in rats. The trial was carried out by an accredited service provider (Intestinal Biotech Development, Lille) in accordance with government guidelines.

For colitis induction, Sprague Dawley rats were anesthetized for 2 h and injected intrarectally with TNBS (Trinitrobenzene sulfonic acid, 80 mg/kg dissolved in 40% ethanol).

The animals were sacrificed 4 days after TNBS injection. DSM 33715 was administered orally at a dose of 10⁹ CFU/ml, with force-feeding starting 14 days before colitis induction and continuing until the day of euthanasia. The animals of the “positive control” group received the Pentasa mixed into their food, at a dose of 150 mg/kg. The anti-inflammatory effect of the products tested was evaluated at the time of sacrifice, following a total treatment period of 18 days.

The variations in weight were monitored during the entire period of the experiment. The length of the colon was evaluated on the day of the autopsy. Macroscopic and microscopic scores were carried out by two different operators to validate the scores. The colon of each rat was examined to evaluate the macroscopic lesions according to the Wallace score. The Wallace score is an evaluation of the macroscopic lesions on a scale of 0 to 10, and this score is based on characteristics reflecting inflammation, such as hyperemia, the thickness of the intestine, and the extent of the ulcers.

The method is described by Wallace J L et al. Gastroenterology. 1992 January; 102(1):18-27. PMID: 1309357 or Wallace J L et al. Inhibition of leukotriene synthesis markedly accelerates healing in a rat model of inflammatory bowel disease. Gastroenterology. 1989 January; 96(1):29-36. PMID: 2535830.

For histological evaluation, based on Ameho criteria, a cut 2 cm above the anal canal was carried out. The sections obtained were stained according to the MGG staining technique (May-Grünwald Giemsa). This classification on a scale of 0 to 6 takes into account the degree of the inflammatory infiltrate, the presence of erosion, ulcer or necrosis, as well as the depth and extent of the lesions. The method is described by Ameho et al., 1997, Gut.

Inflammation was evaluated in the colon by measuring IL-1P production (pro-inflammatory cytokine) (eBioscience kit), as well as the level of Lipocalin-2 (Lcn-2), a neutrophil infiltration marker (Clinisciences) using ELISA quantification. To summarize a distal colon centimeter was collected and homogenized in a Tris-HCl solution containing a cocktail of protease inhibitors (Sigma-Aldrich) by the use of the Precellys homogenizer using ceramic beads (1.4 and 2.8 mm). The samples obtained were centrifuged for 20 min and the supernatants collected and stored at −80° C., until they are used for the ELISAs.

### Results

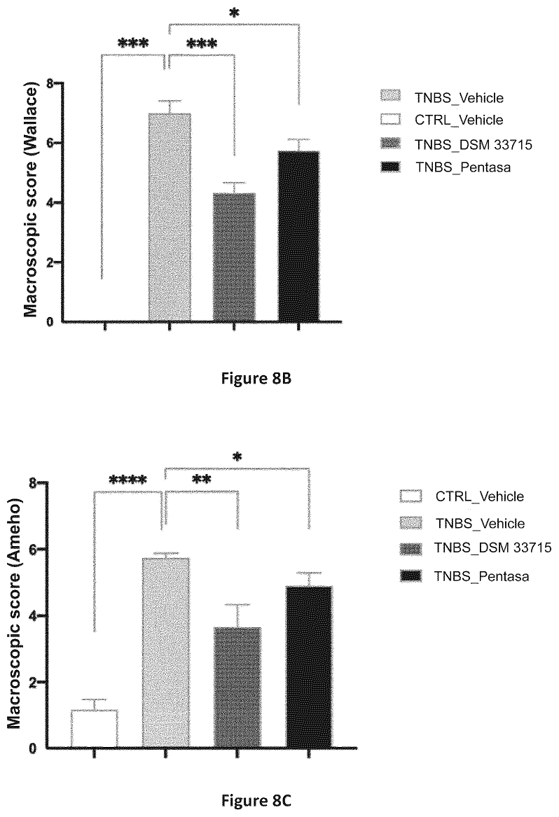

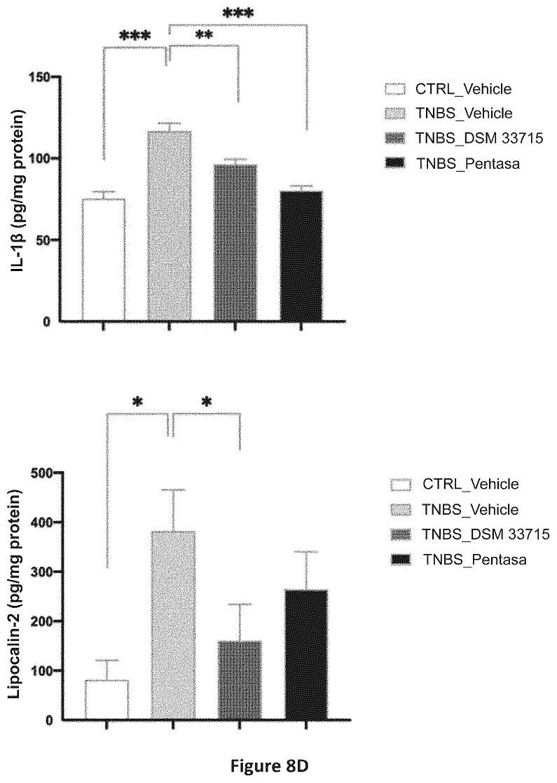

FIG. 8A: The inventors observed a significant decrease in the length of the colon in the group of rats treated with TNBS that received the treatment vehicle relative to the control group that received the vehicle, as expected. A significant increase in the length of the colon was observed in the group of TNBS-treated rats that received the DSM 33715 strain relative to the TNBS-Vehicle group. The DSM 33715 strain has a protective effect on the colon in the TNBS-induced colitis model.

FIG. 8B: The intensity of colonic inflammation and lesions was assessed macroscopically using the Wallace score. A significant increase in Wallace score was observed in rats in the TNBS-Vehicle group compared with the “healthy” rat group, with scores of 7.00+/−0.41 vs. 0L0+1-0.0 respectively, p<0.0001. This result indicates that the rats showed severe colitis 4 days after induction, with an ulcerative or inflammatory site greater than 3 cm of colon (corresponding to a score of 7, on a scale of 0 to 10). A significant reduction in score was observed in the TNBS-Pentasa group and the TNBS-DSM 33715 group. DSM 33715 improved macroscopically observed inflammatory lesions by 38% compared to the TNBS-vehicle group, with Wallace scores of 4.33+1-0.33 vs. 7.00+1-0.41 respectively, p=0.049. As a result, the treatment induces a significant anti-inflammatory effect.

FIG. 8C: A significant improvement in the histological score—intensity of inflammatory lesions—was observed in the group of TNBS rats that were given the positive control Pentasa and the DSM 33715 strain, compared with TNBS rats that were given the vehicle. Pentasa improves the score by 13%, and DSM 33715 by 36%.

FIG. 8D: The level of lipocaline-2, the infiltration marker of neutrophils, is significantly increased in colitic rats (TNBS-Vehicle) compared to a healthy control, confirming the development of severe colitis. A tendency to decrease is observed for Pentasa and a significant reduction in the LCN-2 is observed in the group of TNBS rats treated by the strain DSM 33715.

As expected, a significant increase in IL-1p was measured in the colon of rats in the TNBS-Vehicle group, corrected in part by the treatment with Pentasa, validating the model. Administration of DSM 33715 in colitic rats showed a significant decrease in IL-1P levels, indicating an anti-inflammatory effect of DSM 33715 in acute colitis induced by TNBS in rats.

All these results correlate to demonstrate the anti-inflammatory properties of the DSM 33715 strain, particularly in relation to the reference treatment for this type of pathology, but also in comparison with the reference strain of C. minuta. Thus, the strain according to the invention has a certain therapeutic potential in intestinal dysbiosis and in particular in the prevention and/or treatment of inflammatory diseases.

### Example 9: Comparative Test in an In Vivo Model of Colitis

In the present study, the inventors studied the in vivo effect of the strain according to the invention and of the C. minuta reference strain (DSM22607) in a TNBS-induced colitis model in rats. The test was carried out according to the same protocol as that described in example 8.

As TNBS induction took place after 2 weeks of pretreatment with C. minuta or the control in rats, the macroscopic and microscopic scores were evaluated.

Macroscopic scores (physiological evaluations reflecting inflammation based on hyperemia, thickening of the intestine, diarrhea and the extent of ulcers), change in the body weight and colon weight were analyzed 4 days after TNBS induction.

### Results

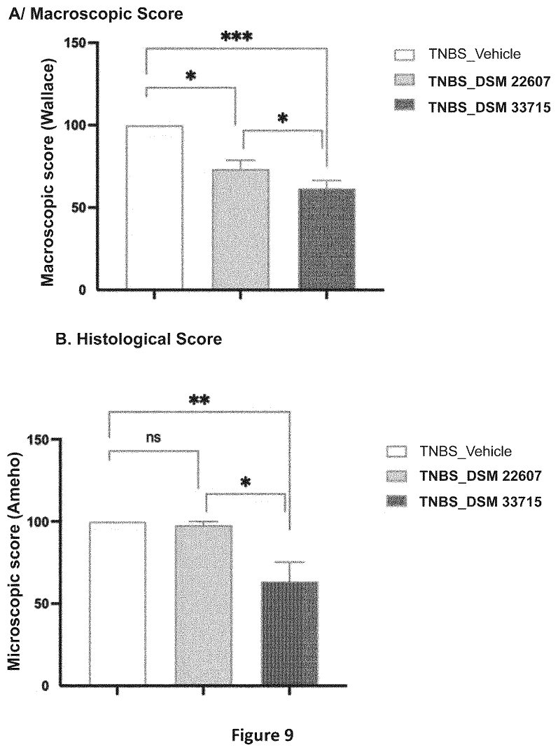

The results are shown in FIG. 9. The results presented in panel A demonstrate the intensity of the inflammation and lesions of the colon which were evaluated at the macroscopic level according to the Wallace score in a model of TNBS-induced acute colitis in rats. This result indicates that the untreated rats had severe colitis 4 days after induction. An anti-inflammatory effect was also observed in the TNBS-DSM 22607 group and more still in the TNBS-DSM 33715 (Invention) group. Thus, the strain DSM 22607 decreases the appearance of the lesions by 27% whereas the strain according to the invention (DSM 33715) decreases them by 38% relative to the TNBS-vehicle group. (Statistics: p=0.02 and p=0.0001 respectively).

The results presented in panel B demonstrate, in this same model of acute colitis in rats, a significant 36% reduction of the histological score of the colon sections in the group of TNBS rats having received the strain according to the invention (DSM 33715) whereas this is not the case in animals that received the treatment with DSM 22607. As the histological score reflects the intensity of inflammatory lesions at the tissue level, this result highlights the superiority of DSM 33715 over DSM 22607 in the treatment of gastrointestinal inflammation.

Thus, all of these results indicate major anti-inflammatory properties of the DSM 33715 strain and an upper effect relative to the DSM 22607 reference strain in the acute colitis model induced by TNBS in rats.

1. A bacterial strain of Christensenella minuta filed as number DSM 33715.

2. A culture supernatant characterized in that it is obtained from the bacterial strain according to claim 1.

3. A composition comprising (i) at least one bacterial strain according to claim 1 and/or (ii) a culture supernatant obtained from the bacterial strain and (iii) at least one acceptable excipient.

4. The composition according to claim 3, characterized in that it is living, dead, attenuated or inactivated.

5. The composition according to claim 4, characterized in that it is in solid, liquid or lyophilized form.

6. The composition of claim 3, wherein said composition being in a form suitable for oral, nasal, parenteral, rectal, sublingual, ocular, auricular, intramuscular, intravenous, inhaled or cutaneous administration.

7. The composition of claim 3, characterized in that it is in the form of a powder, microencapsulated powder, gel capsule, capsule, tablet, pellet, granule, emulsion, suspension, suppository, inhaler, or syrup.

8. The composition of claim 3, characterized in that it comprises at least 50% living bacteria (in number).

9. The composition of claim 3, characterized in that the living bacteria represent between 10⁵ to 10¹¹ CFU.

10. The composition of claim 3, wherein the composition is in the form of a food product, a beverage, a nutraceutical, a food additive, a food supplement or a dairy product.

11. The composition of claim 3, also comprising at least one probiotic and/or at least one bacterium of the family Christensenellaceae and/or an anti-inflammatory agent.

12. The composition of claim 11, further comprising a bacterium selected from Akkermansia muciniphila, Faecalibacterium prausnitzii, Anaerobutiryricum hallii, Hafnia alvei, Roseburia intestinalis, Roseburia hominis, Roseburia faecis, Roseburia inulinivorans, Dysosmobacter welbionis, Oscillospira guillermondii, Lactiplantibacillus plantarum, Lacticaseibacillus casei, Latilactobacillus sakei, Ligilactobacillus salivarius, Limosilactobacillus fermentum, Limosilactobacillus reuteri, Levilactobacillus brevis, Bifidobacterium pseudolongum, Bifidobacterium longum, Bifidobacterium infantis, Bifidobacterium animalis, Bifidobacterium adolescentis, Bifidobacterium bifidum, Bifidobacterium breve, Bifidobacterium lactis, Bifidobacterium stercoris and Bifidobacterium thermophilum.

13. The composition of claim 11, further comprising a pharmaceutical active ingredient selected from mesalamine, olsalazine, budesonide, tofacitinib, filgotinib, upadacitinib, infliximab, adalimumab, golimumab, certolizumab, vedolizumab and ustekinumab.

14. The composition of claim 3, for use thereof as a medicament in humans or animals.

15. The composition of claim 3, for use in the prevention and/or treatment of dysbiosis of microbiota.

16. The composition of claim 3, for use in the prevention and/or treatment of a disease selected from among chronic diseases, metabolic diseases, inflammatory diseases, respiratory function disorders, and cancers.

17. The composition for use according to claim 16, in the prevention and/or treatment of chronic inflammatory bowel diseases.

18. The composition for use according to claim 17, in the prevention and/or treatment of a disease selected from Crohn's disease, ulcerative colitis, hemorrhagic ulcerative rectocolitis, diverticulitis, esophagitis, gastritis, pancreatitis, peptic ulcers, irritable bowel syndrome.
